(19) United States
(12) Patent Application Publication
(10) Pub. No.: US 2024/0349734 A1
(43) Pub. Date: Oct. 24, 2024

(54) RIGID SURFACE TREATMENT AGENT

(71) Applicant: KAO CORPORATION, Tokyo (JP)

(72) Inventors: Takahiro HIOKI, Wakayama-shi, Wakayama (JP); Saki TAKAHIRA, Sumida-ku, Tokyo (JP); Akihito KAWAHARA, Wakayama-shi, Wakayama (JP)

(73) Assignee: KAO CORPORATION, Tokyo (JP)

(21) Appl. No.: 18/683,618

(22) PCT Filed: Aug. 23, 2022
(86) PCT No.: PCT/JP2022/031706

§ 371 Date: Feb. 14, 2024

(30) Foreign Application Priority Data
Aug. 26, 2021 (JP) 2021-137860

(51) Int. Cl. A01N 63/50; C12N 9/64

(52) U.S. Cl. CPC A01N 63/50; C12N 9/6489; C12Y 304/24032

(57) ABSTRACT

Provided are a rigid surface treatment agent capable of imparting an antibacterial property to a rigid surface, and a method for treating a rigid surface using the rigid surface treatment agent. The rigid surface treatment agent comprises, as an active component, a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence.

### FIELD OF THE INVENTION

The present invention relates to a rigid surface treatment agent, and use thereof.

### BACKGROUND OF THE INVENTION

Microorganism contamination on environmental surfaces is a major source of infection with pathogenic bacteria (Non Patent Literature 1). Aside from pathogenic bacteria, microorganism contamination of sinks, drainage ports, laundry tubs or the like is a cause of offensive odors, slime and the like. The microorganism contamination of the surfaces thereof is removed typically by use of ethanol, hypochlorous acid, hot water, a surfactant or the like, and frequent cleaning is required for maintaining a surface free from microorganism contamination. Therefore, a technique for continuously controlling microorganisms on the surfaces of sinks, drainage ports, laundry tubs, medical instruments, ward environments, portable articles, livestock utensils and the like is desired.

As an example of the technique, techniques are well known in which bacteria-suppressing processing for inhibiting proliferation of bacteria is applied to a rigid surface to perform hygiene control. For example, the techniques are commonly used not only for rigid surfaces which are frequently touched by many unspecified people, such as surfaces of public doorknobs, arm rails and hanging straps, but also for any installations and tools used in nursing and care facilities, hospitals and the like, and general daily necessities. The techniques have come into use for private belongings. These techniques are intended to impart a bacteria-suppressing property by kneading a bacteria-suppressing agent in plastic which forms a rigid surface, or applying or spraying to adhere a bacteria-suppressing agent to a rigid surface. As the bacteria-suppressing agent, organic compounds well known to have a bacteria-suppressing action, and particles or ions of metals such as silver and copper are common, but in some techniques, a residual effect of a bactericide is insisted as being bacteria-suppressing effect. Patent Literature 1 discloses a bacteria-suppressing cleaner for rigid surfaces which contains silicone having a specific structure, and a quaternary ammonium compound, a phenol resin, a guanide derivative, an alkyl alcohol and others as bactericides. Patent Literature 2 discloses a bacteria-suppressing property imparting agent using nanoparticulated silver fine particles, and Patent Literatures 3 and 4 disclose an antifouling agent containing a cationic polymer. Patent Literature 5 discloses a bacteria-suppressing composition for rigid surfaces which contains a cationic substance with bacteria-suppressing activity, and Patent Literature 6 discloses a bacteria-suppressing cleansing composition containing catechin and a cationic surfactant.

As a technique using an enzyme, it has been reported that by fixing lysostaphin having Staphylococcus aureus lysing activity to a plastic surface, Staphylococcus aureus killing activity can be imparted to the surface (Non Patent Literature 2). This effect is exhibited simply by bringing a lysostaphin solution and plastic into contact with each other. However, lysostaphin is known to effectively act only on Staphylococcus (Non Patent Literature 3), and a technique for controlling a broader range of microorganisms is necessary. On the other hand, techniques for making a rigid surface bacteria-suppressing using a lysozyme having bacteria-suppressing activity against a broader range of microorganisms have been reported (Non Patent Literatures 4 and 5). However, these techniques require crosslinking treatment by covalent bonding and/or pretreatment of a surface for fixation to the surface, and are applicable in a limited range. Staphylococcus aureus as critical hazardous bacteria is known to have resistance to lysozyme due to its cell wall structure (Non Patent Literature 6).

The β-lytic metalloprotease (beta-lytic metallopeptidase; BLP) which is a protease belonging to the M23A subfamily has been reported to have potent bacteriolytic activity against gram-positive bacteria such as Staphylococcus aureus and Bacillus subtilis (Non Patent Literatures 7 and 8). It has been found that the M23A subfamily protease can be efficiently produced from a culture by introducing a M23A family protease gene into Bacillus bacteria hosts, and culturing the cells (Patent Literature 7).

However, it has not been heretofore reported that the M23A family protease is used for making a rigid surface antibacterial.

(Patent Literature 1) JP-A-2004-532300

(Patent Literature 2) JP-A-2000-178595

(Patent Literature 3) JP-A-2002-60786

(Patent Literature 4) JP-A-2020-152856

(Patent Literature 5) JP-A-2003-510450

(Patent Literature 6) JP-A-2008-195917

(Patent Literature 7) WO 2019/142773

(Non Patent Literature 1) Donskey, Curtis J. American journal of infection control, 2013, 41 (5): S12-S19

(Non Patent Literature 2) Shah, Anjali et al. Antimicrobial agents and chemotherapy, 2004, 48 (7): 2704-2707

(Non Patent Literature 3) Schindler, Ch A. and V. T. Schuhardt, Proceedings of the National Academy of Sciences of the United States of America, 1964, 51 (3): 414-421

(Non Patent Literature 4) Yuan, Shaojun, et al. Langmuir, 2011, 27.6:2761-2774

(Non Patent Literature 5) Yu, Wu-Zhong, et al. Materials & Design, 2018, 139:351-362

(Non Patent Literature 6) Bera, Agnieszka et al. Journal of Bacteriology, 2007, 189 (1): 280-283

(Non Patent Literature 7) Li, Shaoliang et al. The Journal of Biochemistry, 1998, 124 (2): 332-339

(Non Patent Literature 8) Ahmed, Kashfia et al. Journal of Bioscience and Bioengineering, 2003, 95 (1): 27-34

### SUMMARY OF THE INVENTION

The present invention relates to the following 1) to 3).

1) A rigid surface treatment agent comprising, as an active component, a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence.

2) A method for treating a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

3) A method for imparting an antibacterial property to a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

### BRIEF DESCRIPTION OF DRAWINGS

FIG. 1 shows bactericidal activity of BLP and lysostaphin in a solution.

FIG. 2 shows antibacterial activity of BLP for stainless steel.

FIG. 3 shows antibacterial activity of BLP for stainless steel.

FIG. 4 shows antibacterial activity of BLP for stainless steel.

FIG. 5 shows antibacterial activity of BLP for stainless steel.

FIG. 6 shows antibacterial activity of BLP for stainless steel.

FIG. 7 shows antibacterial activity of BLP, lysostaphin and lysozyme for stainless steel.

FIG. 8 shows antibacterial activity of BLP and lysostaphin for plastic.

### DETAILED DESCRIPTION OF THE INVENTION

In the present specification, the term “identity of at least 80%” concerning nucleotide sequences or amino acid sequences refers to an identity of 80% or more, preferably 85% or more, more preferably 90% or more, further more preferably 95% or more, even more preferably 97% or more, even more preferably 98% or more, even more preferably 99% or more.

In the present specification, the identity between nucleotide sequences or amino acid sequences can be calculated by a Lipman-Pearson method (Lipman-Pearson Method; Science, 1985, 227:1435-41). Specifically, the identity can be calculated by performing analysis using the homology analysis (search homology) program of genetic information processing software Genetyx-Win (Ver. 5.1.1; Software Development), where the unit size to compare (ktup) is set to 2.

In the present specification, the “position corresponding to” on an amino acid sequence or a nucleotide sequence can be determined by aligning a target sequence and a reference sequence (for example, an amino acid sequence of SEQ ID NO: 2) so as to give maximum homology to conserved amino acid residues or nucleotides present in each of the amino acid sequences or nucleotide sequences (alignment). The alignment can be performed using a known algorism, and the procedure thereof is known to those skilled in the art. For example, the alignment can be performed by using a Clustal W multiple alignment program (Thompson, J. D. et al, 1994, Nucleic Acids Res., 22:4673-4680) by default. It is also possible to use Clustal W2 and Clustal omega which are revised versions of Clustal W. Clustal W, Clustal W2 and Clustal omega can be used, for example, on the websites of European Bioinformatics Institute: EBI [www.ebi.ac.uk/index/html] and DNA Data Bank of Japan managed by National Institute of Genetics (DDBJ [www.ddbj.nig.ac.jp/Welcome-j.html]). The position of amino acid residue or nucleotide of the target sequence, which are aligned to a position corresponding to an arbitrary position of the reference sequence by the above-described alignment, is taken as a “position corresponding to” the arbitrary position.

In the present specification, the phrase “operable linkage” between a control region and a gene means that the gene and the control region are linked such that the gene can be expressed under the control of the control region. The procedure for “operable linkage” between a gene and a control region is well known to those skilled in the art.

The term “M23A subfamily protease” refers to a protease which has activity of degrading a glycine-glycine bond in a peptide sequence, and which is classified into the M23A subfamily, a metalloprotease subfamily belonging to the M23 family, when classified in accordance with the classification method of the MEROPS database (Rawlings, Neil D., et al. “MEROPS: the database of proteolytic enzymes, their substrates and inhibitors.” Nucleic acids research 42.D1 (2013): D503-D509).

The term “β-lytic metalloprotease (beta-lytic metallopeptidase; BLP)” (MEROPS ID: M23.001) refers to an enzyme also called a β-lytic protease, which is one of proteases belonging to the M23A subfamily.

The present invention relates to provision of a rigid surface treatment agent capable of imparting an antibacterial property to a rigid surface, and a method for treating a rigid surface using the rigid surface treatment agent.

The present inventors found that simply by bringing BLP into contact with a rigid surface, an antibacterial property can be imparted to the surface.

An enzyme polypeptide provided by the present invention can impart an antibacterial property to a rigid surface simply by bringing the polypeptide into contact with the rigid surface. The enzyme can impart an antibacterial property even in the presence of surfactant. The imparted antibacterial property can be maintained even if the rigid surface is washed and dried after the enzyme and the rigid surface are brought into contact with each other.

The polypeptide of the present invention includes BLP, and polypeptides functionally equivalent to BLP, and it is preferable that one of these polypeptides be appropriately selected, and used.

BLP is a polypeptide consisting of an amino acid sequence of SEQ ID NO: 2, which is encoded by the nucleotide sequence from positions 595 to 1134 in SEQ ID NO: 1. BLP has activity of degrading a glycine-glycine bond in a peptide sequence.

Examples of the polypeptide functionally equivalent to BLP include polypeptides which consist of an amino acid sequence having an identity of at least 80% with an amino acid sequence of SEQ ID NO: 2, and have activity of degrading a glycine-glycine bond in a peptide sequence. Preferred examples of the polypeptide functionally equivalent to BLP include polypeptides which consist of an amino acid sequence having an identity of at least 80% with an amino acid sequence of SEQ ID NO: 2, preferably with His at positions corresponding to positions 22, 121 and 123 and Asp at a position corresponding to position 36 in the amino acid sequence of SEQ ID NO: 2, and have activity of degrading a glycine-glycine bond in a peptide sequence.

The presence or absence of the activity of degrading a glycine-glycine bond can be determined by, for example, examining the property of degrading an oligo-glycine peptide, a Fret-GGGGG substrate or the like, but is not limited to this method.

The polypeptide according to the present invention can be extracted or prepared from microorganisms producing the polypeptide or a culture thereof. For example, BLP can be extracted or prepared from Lysobactersp. (NBRC 12725 or NBRC 12726), Achromobacter LyticusM497-1, Lysobacter sp. IB-9374, Lysobacter gummosus DSMZ 6980 or the like, or a culture thereof. The microorganisms can be purchased from official collections of microorganisms.

Microorganisms producing the polypeptide according to the present invention may be cultured under appropriate conditions in a culture medium containing consumable carbon sources, nitrogen sources, metal salts, vitamins and the like. From the thus-obtained microorganisms and culture solution, an enzyme can be collected and prepared by a common method, and subjected to freeze-drying, spray-drying, crystallization and the like to obtain a necessary enzyme form. For example, the enzyme can be collected and prepared from the culture using any of ordinary methods such as separation of microorganisms by centrifugation or filtration, precipitation of an enzyme in the supernatant or filtrate by addition of a salt such as ammonium sulfate or addition of an organic solvent such as ethanol, concentration or desalting with an ultrafiltration membrane, and purification by any of various kinds of chromatography such as ion exchange and gel filtration.

Alternatively, the polypeptide according to the present invention can be produced by a chemical synthetic or biological method with the utilization of the above-described amino acid sequences. For example, the polypeptide according to the present invention can be obtained in accordance with the method disclosed in Patent Literature 7, by culturing Bacillus bacteria transformed to express a polynucleotide encoding a protein prepared by extracting genomic DNA by a conventional method from microorganisms which naturally produce the polypeptide according to the present invention, or extracting RNA from the microorganisms and synthesizing cDNA by reverse transcription, and further introducing a mutation if necessary, and by preparing an objective enzyme from the culture. Examples of the transformed Bacillus bacteria prepared here include Bacillus bacteria obtained by introducing a gene, which is operably linked to a control region and which encodes the polypeptide according to the present invention, into genomes or plasmids of host cells, and Bacillus bacteria having an expression vector introduced in which a target gene is incorporated at an appropriate position.

Here, a “control region” of a gene is a region having a function of controlling intracellular expression of a gene downstream of the region, preferably a function of constitutionally expressing or highly expressing a downstream gene. Specifically, the control region can be defined as a region which is present upstream of a coding region in the gene, and has a function of controlling transcription of the gene by RNA polymerase interaction. Preferably, a control region of a gene refers to a region of about 200 to 600 nucleotides upstream of the coding region in the gene. The control region includes a transcription start control region and/or translation start control region of gene, or a region extending from the transcription start control region to the translation start control region. The transcription start control region includes a promoter and a transcription start point, and the translation start control region is a site corresponding to the Shine-Dalgarno (SD) sequence forming a ribosome binding site together with a start codon (Shine, J., Dalgarno, L., Proc. Natl. Acad. Sci. USA., 1974, 71:1342-1346).

The expression vector containing a gene encoding the polypeptide according to the present invention can be prepared by incorporating a gene encoding the polypeptide according to the present invention into a vector which is capable of stably retaining the gene, and replicating and maintaining the gene in host microorganisms, and enables the polypeptide to be stably expressed. Examples of the vector include shuttle vectors such as pHA3040SP64, pHSP64R or pASP64 (JP-B-3492935), pHY300PLK (expression vector capable of transforming both Escherichia coli and Bacillus subtilis; Jpn J Genet, 1985, 60:235-243), and pAC3 (Nucleic Acids Res, 1988, 16:8732); and plasmids usable for transformation of Bacillus bacteria, such as pUB110 (J Bacteriol, 1978, 134:318-329), and pTA10607 (Plasmid, 1987, 18:8-15). It is also possible to use plasmids derived from E. coli (for example, pET22b (+), pBR322, pBR325, pUC57, pUC118, pUC119, pUC18, pUC19, pBluescript and the like).

The host Bacillus bacterium can be transformed using a protoplast method, a competent cell method, an electroporation method, or the like. The host Bacillusbacterium is preferably Bacillus subtilis or a mutant strain thereof. Examples thereof include Bacillus subtilis strains which produces a reduced amount of extracellular proteases while having sufficient M23A maturing ability.

The obtained transformant may be cultured under appropriate conditions in a culture medium containing consumable carbon sources, nitrogen sources, metal salts, vitamins and the like. From the thus-obtained culture, an enzyme can be collected and prepared by a common method, and further subjected to freeze-drying, spray-drying, crystallization and the like to obtain a necessary enzyme form. For example, the enzyme can be collected and prepared from the culture using any of ordinary methods such as separation of recombinant microorganisms by centrifugation or filtration, precipitation of an enzyme in the supernatant or filtrate by addition of a salt such as ammonium sulfate or addition of an organic solvent such as ethanol, concentration or desalting with an ultrafiltration membrane, and purification by any of various kinds of chromatography such as ion exchange and gel filtration.

Alternatively, the polypeptide according to the present invention can be prepared from an enzyme composition containing the polypeptide, or the like. For example, BLP can be prepared from Achromopeptidase. Achromopeptidase is a bacteriolytic enzyme derived from Lysobacter enzymogenes, and contains BLP. Achromopeptidase is commercially available from Wako Pure Chemical Industries, Ltd.

As shown in Examples described later, the polypeptide according to the present invention, for example, BLP has an antibacterial action for a rigid surface, and can impart an antibacterial property to a rigid surface by bringing BLP into contact with the rigid surface. BLP is equivalent in Staphylococcus aureuskilling action in a solution to lysostaphin described in Non Patent Literature 2, but unexpectedly, the antibacterial action exhibited by bringing BLP into contact with a rigid surface is higher than the antibacterial action exhibited by bringing lysostaphin into contact with a rigid surface. Surprisingly, the antibacterial action of BLP for a rigid surface is maintained at a high level even if a rigid surface is washed after BLP and the rigid surface are brought into contact with each other, that is, the rigid surface comes into contact with water, or the rigid surface is dried. Further, the antibacterial action of BLP for a rigid surface is recognized regardless of the material of the rigid surface, for example, whether the rigid surface is a stainless steel surface or a plastic surface, and even in the presence of a surfactant.

Therefore, the polypeptide according to the present invention is useful as a rigid surface treatment enzyme for imparting an antibacterial property to the rigid surface, and can be a rigid surface treatment agent, preferably a rigid surface treatment agent for imparting an antibacterial property. Alternatively, the polypeptide according to the present invention can be used for producing a rigid surface treatment agent, preferably a rigid surface treatment agent for imparting an antibacterial property.

The polypeptide according to the present invention can be used for rigid surface treatment, preferably rigid surface treatment for imparting an antibacterial property. For example, by bringing the polypeptide according to the present invention into contact with a rigid surface of interest, an antibacterial property can be imparted to the rigid surface.

In the present invention, the term “antibacterial” includes all of the concepts of suppression of attachment of bacteria on a rigid surface, suppression of remaining of bacteria on a rigid surface, the term “bactericidal” which means that bacteria are killed on a rigid surface, the term “sterile”, the term “bacteria-suppressing” which means that development, growth and proliferation of bacteria are suppressed on a rigid surface, the term “bacteriostatic”, and the term “bacterial control”. The present inventors found that BLP is particularly excellent in terms of suppression of remaining or attachment of bacteria on a rigid surface.

The antibacterial activity can be evaluated using a well-known method in the art. For example, it can be evaluated by immersing a test piece having a rigid surface in a solution containing an objective polypeptide for a predetermined time, thereby bringing the test piece into contact with the polypeptide; immersing the test piece in a test solution containing test bacteria for a predetermined time, thereby bringing the test piece into contact with the test bacteria; then extracting the test bacteria from the test piece and culturing the test bacteria in an appropriate solid culture medium; and counting the number of generated colonies to calculate the number of viable bacteria attached to the test piece.

In the present invention, bacteria against which the term “antibacterial” can be used are not limited, and are preferably gram-positive bacteria. Examples of the gram-positive bacteria include Staphylococcus bacteria such as Staphylococcus aureus and Staphylococcus epidermidis; Micrococcus bacteria such as Micrococcus luteus; Streptococcus bacteria such as Streptococcus pneumoniae, Streptococcus viridans, Streptococcus pyogenes and Streptococcus agalactiae; Enterococcus bacteria such as Enterococcus faecalis; Bacillus bacteria such as Bacillus anthracis; Clostridium bacteria such as Clostridium tetani, Clostridium perfringens and Clostridium botulinum; Corynebacterium bacteria such as Corynebacterium diphtheriae; and Listeria bacteria such as Listeria monocytogenes. Of these, Staphylococcusbacteria and Micrococcus bacteria are more preferable, and Staphylococcus aureus and Micrococcus luteus are further more preferable.

The rigid surface treatment agent of the present invention may be in the form of the polypeptide of the present invention used alone, or in the form of an enzyme composition containing the polypeptide. The enzyme composition may be a solid composition in the form of powder or the like, or a liquid composition. The enzyme composition may be in an undiluted form, or in a diluted form. In the case of the undiluted form, the enzyme composition is used for rigid surface treatment without being diluted. In the case of the diluted form, the enzyme composition is diluted with an appropriate medium such as water such that the content of the polypeptide according to the present invention after dilution is within a range described below, followed by use for rigid surface treatment.

Preferably, the rigid surface treatment agent is a product or preparation for use in, for example, antibacterial treatment for a rigid surface of a non-living object on which bacteria are or may be present, or to which bacteria may be attached, or the rigid surface treatment agent is used as an antibacterial material in the product or preparation.

Examples of the rigid surface include rigid surfaces of non-living objects on which bacteria are or may be present, or to which bacteria may be attached, for example, rigid surfaces of counters, sinks, lavatories, toilets, laundry tubs, bathtubs, shower stands, floors, windows, doors, doorknobs, walls, sewage ports, pipes and the like; rigid surfaces of various instruments and food processing devices, tools, sundries and the like, for example, kitchenware, furniture, phones, toys, medical instruments, livestock instruments and food processing instruments,; and rigid surfaces which come into contact with water from a water cooling tower of air-conditioning equipment in buildings. Examples of the material of the rigid surface include plastic (including silicone resin), metal, pottery, wood, glass, and combinations thereof. Plastic, metal or a combination thereof is preferable, and plastic, stainless steel or a combination thereof is more preferable.

The rigid surface is preferably in an environment where the rigid surface comes into contact with water on a regular or irregular basis, or in an environment where the rigid surface is dried on a regular or irregular basis. The antibacterial action of the rigid surface treatment agent of the present invention for a rigid surface is maintained even in the above-mentioned environment. Here, the term “environment” refers to an external condition surrounding the rigid surface, and includes not only naturally occurring environments but also intentionally created environments. Thus, the “rigid surface in an environment where the rigid surface comes into contact with water” can be a rigid surface which may come into contact with water in a natural state of being, or a rigid surface which may be caused to come into contact with water in accordance with the user's intention, and the “rigid surface in an environment where the rigid surface is dried” can be a rigid surface which may be dried in a natural state of being, or a rigid surface which may be dried in accordance with user's intention.

The form of the above-described product or preparation can be a solution, an emulsion, a cream, a lotion, a paste, a jelly, a sheet (supporting a substrate), an aerosol, a spray, an oil, a gel, or the like, but is not limited thereto.

The product or preparation may contain a bacteria-suppressing substance such as hypochlorous acid, hydrogen peroxide or a silver ion compound, a cationic bacteria-suppressing agent (benzethonium chloride or the like), a bactericide (triclosan, isopropylmethylphenol or the like), ethanol, a surfactant and the like as appropriate in addition to the polypeptide according to the present invention, and can be prepared through a conventional method by appropriately blending additives such as a chelating agent, a humectant, a lubricant, a builder, a buffering agent, an abrading agent, an electrolyte, a bleaching agent, a perfume, a dye, a foam control agent, a corrosion inhibitor, essential oil, a thickener, a pigment, a gloss improving agent, an enzyme other than the polypeptide according to the present invention, a cleansing agent, a solvent, a dispersant, a polymer, silicone and a hydrotropic substance.

The content of the polypeptide according to the present invention in the rigid surface treatment agent of the present invention can be appropriately determined depending on a form of an enzyme composition. For example, the content of the polypeptide according to the present invention is preferably 0.00001 mass % or more, more preferably 0.0002 mass % or more, further more preferably 0.0005 mass % or more, further more preferably 0.001 mass % or more, further more preferably 0.005 mass % or more, further more preferably 0.01 mass % or more, and preferably 20 mass % or less, more preferably 5 mass % or less, further more preferably 2 mass % or less, with respect to the total mass of the composition. The numerical range of the content of the polypeptide according to the present invention is preferably from 0.00001 to 20 mass %, more preferably from 0.0002 to 5 mass %, further more preferably from 0.0005 to 2 mass %, further more preferably from 0.001 to 2 mass %, further more preferably from 0.005 to 2 mass %, further more preferably from 0.01 to 2 mass %.

The rigid surface treatment agent of the present invention is used by bringing the agent into contact with a rigid surface, and can impart an antibacterial property to the rigid surface. The contact time is preferably 10 seconds or more, more preferably 1 minute or more, further more preferably 5 minutes or more, even more preferably 10 minutes or more, from the viewpoint of imparting an antibacterial property. The upper limit of the contact time is not limited, and the rigid surface treatment agent may be allowed to stand as it is after contact, but when washing or the like is performed after contact, the contact time is preferably 3 hours or less, more preferably 1 hour or less, further more preferably 30 minutes or less, from the viewpoint of workloads. The contact time is preferably 10 seconds or more and 3 hours or less, more preferably 1 minute or more and 1 hour or less, further more preferably from 5 to 30 minutes, even more preferably from 10 to 30 minutes. The contact means is not limited, and may be any of methods such as a method in which the rigid surface treatment agent is applied to a rigid surface, a method in which a rigid surface is immersed in the rigid surface treatment agent, a method in which the rigid surface treatment agent is sprayed or sprinkled on a rigid surface in a state of being atomized with an atomization apparatus such as a pump spray, an aerosol, a pressurized liquid spray or a pressurized air atomization spray apparatus, a method in which a rigid surface is wiped with a sheet, a gauze, a towel, a hand towel, a tissue, a wet tissue or the like impregnated with the rigid surface treatment agent, or a method in which the rigid surface treatment agent placed upstream is gradually released with flowing water to come into contact with a rigid surface in the downstream.

The concentration of the polypeptide according to the present invention in bringing the rigid surface treatment agent of the present invention into contact with a rigid surface is preferably 1 ppm or more, more preferably 5 ppm or more, further more preferably 10 ppm or more, from the viewpoint of imparting an antibacterial property. The upper limit of the concentration of the polypeptide is not limited, and is preferably 1,000 ppm or less, more preferably 500 ppm or less, further more preferably 100 ppm or less. The numerical range of the concentration is preferably from 1 to 1,000 ppm, more preferably from 5 to 500 ppm, further more preferably from 10 to 100 ppm.

In another aspect, the present invention provides a rigid surface treatment method using the polypeptide according to the present invention. In still another aspect, the present invention provides a method for imparting an antibacterial property to a rigid surface using the polypeptide according to the present invention. The method includes bringing the polypeptide according to the present invention or an enzyme composition containing the polypeptide into contact with the rigid surface.

The mode of contact between the polypeptide according to the present invention and a rigid surface may be appropriately selected depending on a shape of the rigid surface, and a type of material thereof, and the treatment time, and the amount of the enzyme used can be arbitrarily set depending on a mode of treatment. For example, a solution containing the polypeptide according to the present invention is applied, sprayed or sprinkled on a rigid surface, and left to stand for a certain time (for example, 10 seconds or more and 3 hours or less), or a rigid surface is immersed in the solution, and left to stand for a certain time (for example, 10 seconds or more and 3 hours or less). The rigid surface after contact may be washed or rinsed with a medium such as water, and may be dried. The number of times of washing is not limited, and is, for example, at least 1, and preferably 4 or less, more preferably 2 or less. The mode of drying is not limited, and may be natural drying, or drying by heating.

Regarding the embodiments described above, the present invention further discloses the following aspects.

<1> A rigid surface treatment agent comprising, as an active component, a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence.

<2> The rigid surface treatment agent according to <1>, which is used for imparting an antibacterial property.

<3> The rigid surface treatment agent according to <1> or <2>, wherein the rigid surface is preferably a plastic surface, a metal surface, or a combination thereof, more preferably a plastic surface, a stainless steel surface, or a combination thereof.

<4> The rigid surface treatment agent according to any one of <1> to <3>, wherein the antibacterial property is against gram-positive bacteria.

<5> The rigid surface treatment agent according to any one of <1> to <4>, wherein the rigid surface is in an environment where the rigid surface comes into contact with water on a regular or irregular basis.

<6> The rigid surface treatment agent according to any one of <1> to <4>, wherein the rigid surface is in an environment where the rigid surface is dried on a regular or irregular basis.

<7> A method for treating a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

<8> A method for imparting an antibacterial property to a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

<9> The method according to <7> or <8>, wherein the rigid surface is preferably a plastic surface, a metal surface, or a combination thereof, more preferably a plastic surface, a stainless steel surface, or a combination thereof.

<10> The method according to any one of <7> to <9>, comprising a step of bringing the polypeptide or the enzyme composition comprising the polypeptide into contact with the rigid surface for preferably 10 seconds or more, more preferably 1 minute or more, further more preferably 5 minutes or more, even more preferably 10 minutes or more.

<11> The method according to any one of <7> to <10>, comprising a step of bringing the polypeptide or the enzyme composition comprising the polypeptide into contact with the rigid surface at a concentration of preferably 1 ppm or more, more preferably 5 ppm or more, further more preferably 10 ppm or more in terms of the polypeptide.

<12> The method according to any one of <7> to <11>, comprising a step of applying, spraying or sprinkling a solution comprising the polypeptide or the enzyme composition comprising the polypeptide on the rigid surface.

<13> The method according to any one of <7> to <11>, comprising a step of immersing the rigid surface in a solution comprising the polypeptide or the enzyme composition comprising the polypeptide.

<14> The method according to any one of <8> to <13>, wherein the antibacterial property is against gram-positive bacteria.

<15> The method according to any one of <7> to <14>, wherein the rigid surface is in an environment where the rigid surface comes into contact with water on a regular or irregular basis.

<16> The method according to any one of <7> to <14>, wherein the rigid surface is in an environment where the rigid surface is dried on a regular or irregular basis. <17> Use of a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, for producing a rigid surface treatment agent, preferably a rigid surface treatment agent for imparting an antibacterial property.

<18> Use of a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, for rigid surface treatment, preferably for imparting an antibacterial property to a rigid surface.

<19> The use according to <17> or <18>, wherein the rigid surface is in an environment where the rigid surface comes into contact with water on a regular or irregular basis.

<20> The use according to <17> or <18>, wherein the rigid surface is in an environment where the rigid surface is dried on a regular or irregular basis.

### EXAMPLES

### Example 1

### (1) Preparation of Enzyme

BLP consisting of an amino acid sequence of SEQ ID NO: 2 was prepared by performing culturing and purification by a method described in JP-A-2020-182945, Example 1. Lysozyme (FUJIFILM Wako Pure Chemical Corporation, 129-06723) and lysostaphin (FUJIFILM Wako Pure Chemical Corporation, 120-06611) were dissolved in 20 mM Tris-HCl (pH 7.5). DC Protein Assay Kit (Bio-Rad Laboratories, Inc.) was used for measuring the concentration of an enzyme solution. BSA Standard Solution (FUJIFILM Wako Pure Chemical Corporation) was used as a standard solution for calculating the mass of protein.

### (2) Bactericidal Test

Staphylococcus aureus NCTC 8325 was used as test bacteria. SCD Culture Medium “Daigo” for general bacteria tests (FUJIFILM Wako Pure Chemical Corporation) was used as a SCD liquid culture medium, SCD Agar Culture Medium “Daigo” for general bacteria tests (FUJIFILM Wako Pure Chemical Corporation) was used as a SCD agar culture medium, LP Diluting Liquid “Daigo” (FUJIFILM Wako Pure Chemical Corporation) was used as a LP diluting liquid, and 20 mM Tris-HCl (pH 7.5) was used as a buffer. A buffer containing each enzyme (BLP, lysostaphin) at a final concentration of 1 ppm was used as a test solution. Test bacteria cultured with shaking overnight at 37° C. in the SCD liquid culture medium were collected, washed and resuspended with the buffer, and adjusted to 10⁸⁻⁹ CFU/mL. To 500 μL of each test solution was added 5 μL of the bacteria solution, and the mixture was incubated at 30° C. for 30 minutes. The test solution was serially diluted with the LP diluting liquid, and 100 μL of each of the diluted solutions was applied to the SCD agar culture medium. Incubation was performed at 37° C. for 24 hours, and colonies were then counted to calculate the number of viable bacteria contained in 1 mL of the test solution (CFU/mL).

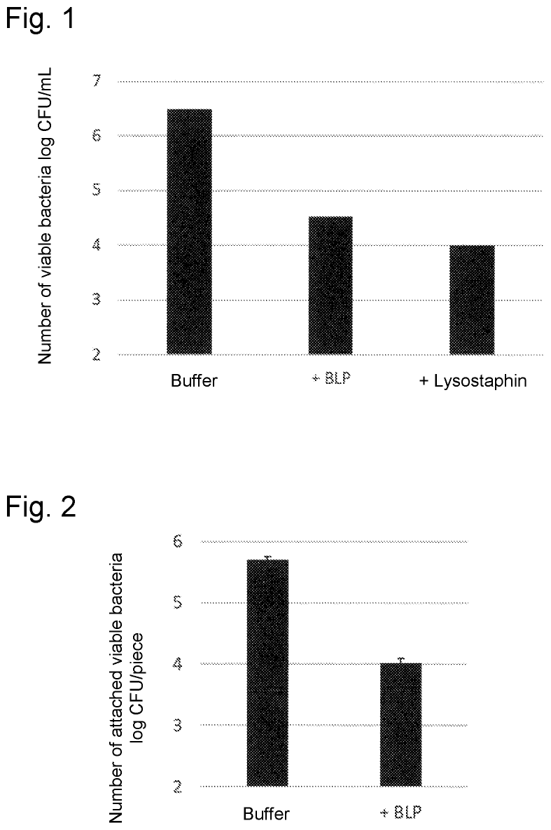

BLP and lysostaphin exhibited the same level of Staphylococcus aureus killing activity in the buffer (FIG. 1).

### (3) Test on Suppression of Attachment of Bacteria to Stainless Steel (Making Stainless Steel Antibacterial by BLP)

Micrococcus luteus was used as test bacteria. As a buffer, 20 mM Tris-HCl (pH 7.5) was used. To each of the wells of a 12-hole polystyrene plate (CORNING, 351143), 1 mL of a BLP solution diluted to 10 ppm with the buffer was added. One SUS430 test piece of 1×15×15 mm (Engineering Test Service Co., Ltd.) was put in each well, and immersed at room temperature for 10 minutes. The test pieces were drained, and transferred to a new 12-hole plate having 2 mL of the buffer added in each well, and the plate was lightly shaken for 2 minutes. Again, the test pieces were drained, and transferred to a new 12-hole plate having 2 mL of the buffer added in each well, and the plate was lightly shaken for 2 minutes, followed by collection of the test pieces. Test bacteria cultured with shaking overnight at 37° C. on the SCD agar culture medium were collected, and washed and resuspended with the buffer, and adjusted to about 10⁸ CFU/mL. To each well of a new 12-hole plate, 1.5 mL of the bacteria solution was added, and one test piece treated by immersion in the enzyme was put in each well. The test pieces were left to stand at room temperature for 15 minutes, then drained, and transferred to a new 12-hole plate having 2 mL of the buffer added in each well, and the plate was lightly shaken for 1 minute. Again, the test pieces were drained, and transferred to a new 12-hole plate having 2 mL of the buffer added in each well, and the plate was lightly shaken for 1 minute. One test piece was put in each 50 mL tube containing 7 mL of a LP diluting liquid, and subjected to ultrasonication for 30 minutes to extract bacteria. The extract was serially diluted with the LP diluting liquid, and 100 μL of each of the diluted solutions was applied to the SCD agar culture medium. Incubation was performed at 37° C. for 24 hours, and colonies were then counted to calculate the number of viable bacteria attached to one test piece (CFU/piece).

The test piece immersed in BLP in advance had a significantly smaller number of attached viable bacteria as compared to the test piece immersed only in the buffer (FIG. 2).

### (4) Test on Suppression of Attachment of Bacteria to Stainless Steel (Effect of Time of Immersion in BLP)

A test on suppression of attachment of bacteria was conducted in the same manner as in (3) except that the time of immersion of the test piece in the enzyme solution was changed to 1, 5, 10 and 30 minutes.

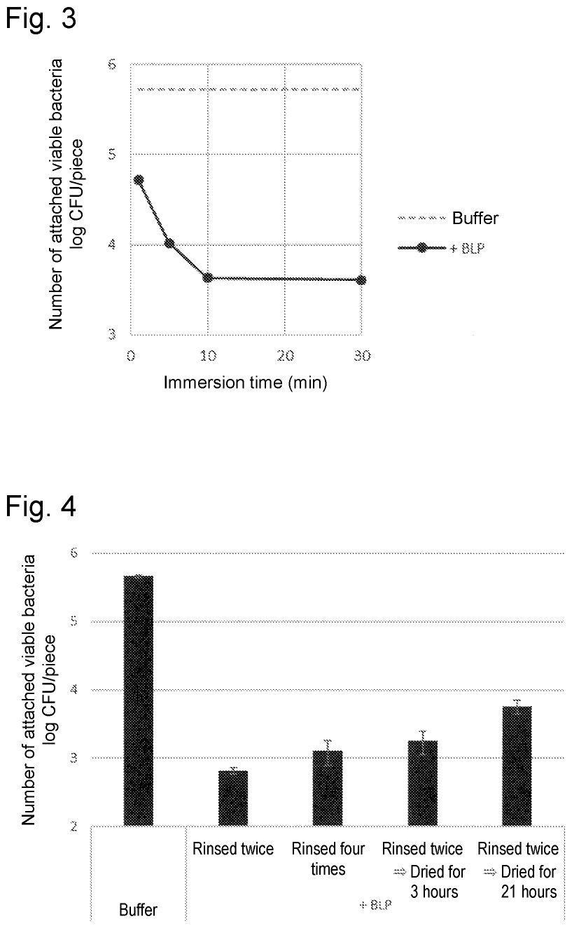

Even in the case of immersion for 1 minute, BLP reduced the number of attached viable bacteria by about 90% as compared to immersion only in the buffer (FIG. 3).

### (5) Test on Suppression of Attachment of Bacteria to Stainless Steel (Resistance of Enzyme Remaining on Surface)

A test on suppression of attachment of bacteria was conducted in the same manner as in (3) except that the enzyme concentration during immersion was changed to 20 ppm, and as test pieces after immersion in the enzyme, those treated under the following four conditions were used.

Condition 1: Rinsed twice (the same condition as in (3)).

Condition 2: Rinsed four times.

Condition 3: The test piece is rinsed twice, and then dried at room temperature for 3 hours.

Condition 4: The test piece is rinsed twice, and then dried at room temperature for 21 hours.

The test pieces immersed in BLP maintained an effect of reducing the number of attached viable bacteria even after rinsing four times and drying for 21 hours (FIG. 4).

(6) Test on Suppression of Attachment of Bacteria to Stainless Steel (Effect on Staphylococcus aureus)

A test on suppression of attachment of bacteria was conducted in the same manner as in (3) except that the test bacteria were changed to Staphylococcus aureus NCTC 8325, and the enzyme concentration during immersion was changed to 20 ppm.

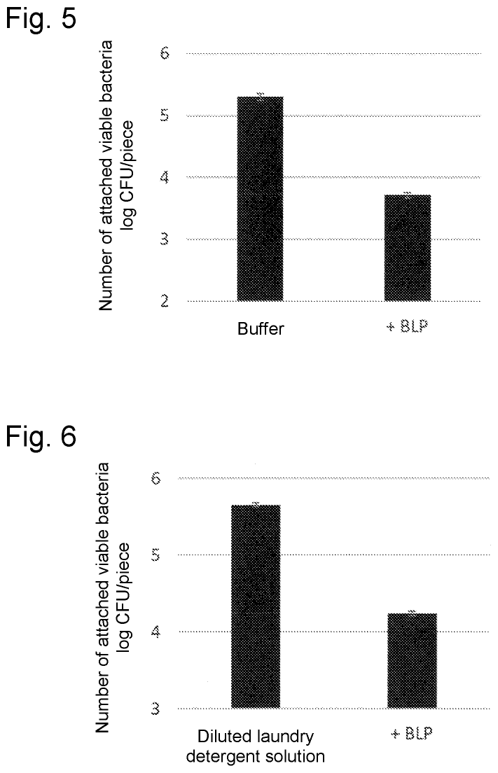

The test piece immersed in BLP also exhibited an effect of reducing the number of attached viable bacteria on Staphylococcus aureus (FIG. 5).

(7) Test on Suppression of Attachment of Bacteria to Stainless Steel (Effect on Staphylococcus aureus)

A test on suppression of attachment of bacteria was conducted in the same manner as in (3) except that the buffer was changed to a solution obtained by diluting a commercially available laundry detergent (Attack ZERO, Kao Corporation) with tap water by a factor of 3,000.

The effect of reducing the number of attached viable bacteria by BLP was also maintained in an aqueous solution containing a surfactant (FIG. 6).

(8) Test on Suppression of Attachment of Bacteria to Stainless Steel (Performance Comparison with Other Bacteriolytic Enzymes)

A test on suppression of attachment of bacteria was conducted in the same manner as in (3) except that the test bacteria was changed to Staphylococcus aureus NCTC 8325, the enzyme concentration during immersion was changed to 20 ppm, and BLP, lysostaphin or lysozyme was used as the enzyme.

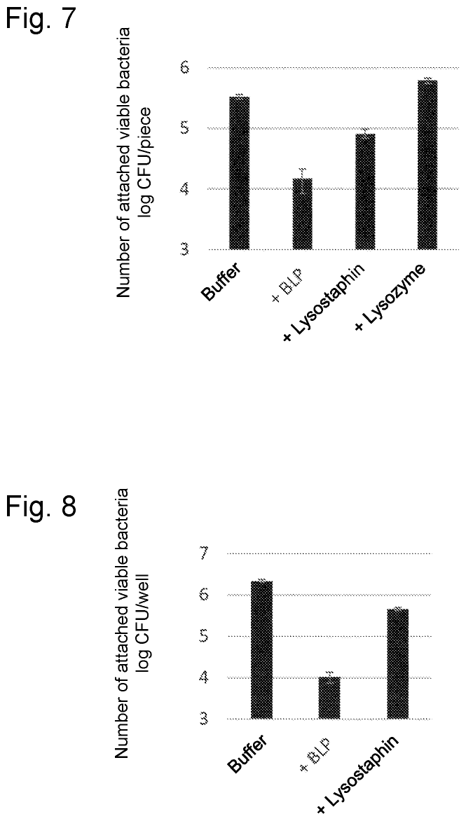

BLP exhibited the greatest effect of reducing the number of attached viable bacteria (FIG. 7).

### (9) Test on Suppression of Attachment of Bacteria to Plastic

Staphylococcus aureus NCTC 8325 was used s test bacteria. As a buffer, 20 mM Tris-HCl (pH 7.5) was used. To each of the wells of a 12-hole polystyrene plate (CORNING, 351143), 1 mL of an enzyme solution diluted to 10 ppm with the buffer was added, followed by immersion at room temperature for 10 minutes. The solution in the wells was all removed with a pipette, 2 mL of the buffer was added to each well, and the plate was lightly shaken for 2 minutes. Again, the solution in the wells was all removed with a pipette, 2 mL of the buffer was added to each well, the plate was lightly shaken for 2 minutes, and the solution in the wells was then all removed with a pipette. The test bacteria cultured with shaking overnight at 37° C. on the SCD agar culture medium were collected, washed and resuspended with the buffer, and adjusted to 10⁸ CFU/mL. To each well of an enzyme-treated 12-hole plate, 1.5 mL of the bacteria solution was added. The bacteria solution was left to stand at room temperature for 15 minutes, the solution in the wells was then all removed with a pipette, 2 mL of the buffer was added to each well, and the plate was lightly shaken for 2 minutes. Again, the solution in the wells was all removed with a pipette, 2 mL of the buffer was added to each well, and the plate was lightly shaken for 2 minutes. The solution in the wells was all removed with a pipette, 2 mL of a LP diluting liquid was added to each well, hermetically sealed, and subjected to ultrasonication for 30 minutes to extract bacteria. The extract was serially diluted with the LP diluting liquid, and 100 μL of each of the diluted solutions was applied to the SCD agar culture medium. Incubation was performed at 37° C. for 24 hours, and colonies were then counted to calculate the number of viable bacteria attached to each well (CFU/well).

BLP also exhibited the effect of reducing the number of attached viable bacteria for polystyrene, and the effect of BLP was higher than the effect of lysostaphin which is shown in Non Patent Literature 2 (FIG. 8).

1. A method for treating a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

2. The method according to claim 1, wherein the rigid surface is in an environment where the rigid surface comes into contact with water on a regular or irregular basis.

3. The method according to claim 1, wherein the rigid surface is in an environment where the rigid surface is dried on a regular or irregular basis.

4. A method for imparting an antibacterial property to a rigid surface, comprising a step of bringing a polypeptide which consists of an amino acid sequence of SEQ ID NO: 2, or a polypeptide which consists of an amino acid sequence having an identity of at least 80% with the amino acid sequence of SEQ ID NO: 2, and has activity of degrading a glycine-glycine bond in a peptide sequence, or an enzyme composition comprising the polypeptide into contact with the rigid surface.

5. The method according to claim 4, wherein the rigid surface is in an environment where the rigid surface comes into contact with water on a regular or irregular basis.

6. The method according to claim 4, wherein the rigid surface is in an environment where the rigid surface is dried on a regular or irregular basis.


SEQUENCE LISTING (2 sequences)

SEQ ID NO: 1 - DNA, 1134 bp, Achromobacter lyticus
  source 1..1134: mol_type = genomic DNA
atgaaaaaaatctcaaaagctggtctgggactggctctggtctgtgctctggcgacgattggaggcaacgcatctgctcagggacatggattaagcggcgaagatctggtttactcttacgatgaaatgtttgattttgatatcgatgcctacctggcaaaacatgcgccgcatctgcataaacatagcgaagaaatctctcattgggccggatattctggcatttcaccgaaagttcttatcgcattaatggaacaacagtcaggagctgtgagcgccaaaagagcaacaaatcgcccgtttggcaaacttgccagagcagatggatttggcgcccaaacacgcgaagtggcgttagctctgagagaatctctttatgaacgcgatccggatggagccaaaggcccggtcacattagccagagcaaacccgctgcaggcactttttgaacgctcaggagataatgaaccggcagcggctttaagaggagatggcgaatttcaacttgtctacggcagattatttaacgaaccgcgccaggcaaaagccgcaagcgatagatttgcgaaagctggaccggatgttcaaccgttatctccgaatggactgcttcagtttccgtttccgagaggcgcatcttggcatgtgggcggagctcatacaaacacaggatcaggcaattatccgatgtcaagcctggatatgtcaagaggcggaggctggggaagcaatcaaaacggcaattgggtttcagcgagcgcggctggatcttttaaacgccattcttcatgctttgctgaaattgttcatacaggcggctggtcaacaacatactaccatctgatgaacatccagtacaatacaggcgcgaacgttagcatgaatacagccatcgcaaacccggctaatacacaagcgcaggctctgtgcaacggaggccaaagcacaggaccgcatgaacattggtcactgaaacagaacggctcattttaccatctgaacggaacatacctttcaggctatagaatcacagcgacaggcagctcttatgatacaaattgtagccgcttttatttgacaaaaaatggacagaactactgctatggttattatgtgaatcctggaccgaactaa

SEQ ID NO: 2 - protein, 179 aa, Achromobacter lyticus
SPNGLLQFPFPRGASWHVGGAHTNTGSGNYPMSSLDMSRGGGWGSNQNGNWVSASAAGSFKRHSSCFAEIVHTGGWSTTYYHLMNIQYNTGANVSMNTAIANPANTQAQALCNGGQSTGPHEHWSLKQNGSFYHLNGTYLSGYRITATGSSYDTNCSRFYLTKNGQNYCYGYYVNPGPN